(19) United States
(12) Patent Application Publication
(10) Pub. No.: US 2015/0157618 A1
(43) Pub. Date: Jun. 11, 2015

(54) Stabilized pharmaceutical compositions of dabigatran and process for preparation thereof

(71) Applicants: Chandrashekhar Kandi, Hyderabad (IN); Nagaprasad Vishnubhotla, Hyderabad (IN); Sreekanth Manikonda, Hyderabad (IN); Anil Kumar Reddy, Hyderabad (IN); Sivakumaran Meenakshisunderam, Hyderabad (IN); Arjuna Rao Panchada, Hyderabad (IN)

(72) Inventors: Chandrashekhar Kandi, Hyderabad (IN); Nagaprasad Vishnubhotla, Hyderabad (IN); Sreekanth Manikonda, Hyderabad (IN); Anil Kumar Reddy, Hyderabad (IN); Sivakumaran Meenakshisunderam, Hyderabad (IN); Arjuna Rao Panchada, Hyderabad (IN)

(21) Appl. No.: 14/544,126

(22) Filed: Nov. 26, 2014

(30) Foreign Application Priority Data
Nov. 26, 2013 (IN) IN 5441/CHE/2013

(51) Int. Cl. A61K 31/4439; A61K 9/48; A61K 9/20

(52) U.S. Cl. CPC A61K 31/4439; A61K 9/20; A61K 9/2095; A61K 9/4808

(57) ABSTRACT

Stabilized pharmaceutical compositions comprising a thrombin inhibitor as an active agent(s), process of preparation and method of using the same are provided. The present invention also relates to stabilized pharmaceutical compositions comprising dabigatran etexilate, or pharmaceutically acceptable salts, esters, hydrates and solvates thereof, process of preparation and method of using the same.

### CROSS REFERENCE TO RELATED APPLICATIONS

This application claims priority from an Indian Patent Application IN 5441/CHE/2013 filed on Nov. 26, 2013

### FIELD OF THE INVENTION

The present invention relates to stabilized pharmaceutical compositions comprising a thrombin inhibitor as an active agent(s), process of preparation thereof, and method of using the same. Particularly the present invention relates to stabilized pharmaceutical compositions comprising dabigatran etexilate, or pharmaceutically acceptable salts, esters, derivatives, hydrates, polymorphs, and solvates thereof, process of preparation and method of using the same.

### BACKGROUND OF THE INVENTION

Anticoagulants are substances that prevent coagulation; that is, they stop blood from clotting. Anticoagulants are widely used in human therapy as a medication for thrombotic disorders, for example primary and secondary prevention of deep vein thrombosis, pulmonary embolism, myocardial infarctions and strokes in those who are predisposed.

An important class of oral anticoagulants acts by antagonizing the effects of vitamin K, for example, the coumarins which include warfarin. A second class of compounds inhibits coagulation indirectly via a cofactor such as antithrombin III or heparin cofactor II. This includes several low molecular weight heparin products which catalyse the inhibition of predominantly factor Xa (and to a lesser degree thrombin) via antithrombin III (bemiparin, certoparin, dalteparin, enoxaparin, nadroparin, parnaparin, reviparin, tinzaparin), smaller chain oligosaccharides (fondaparinux, idraparinux) inhibit only factor Xa via antithrombin III. Heparinoids (danaparoid, sulodexide, dermatan sulfate) act via both cofactors and inhibit both factor Xa and thrombin. A third class represents the direct inhibitors of coagulation. Direct factor Xa inhibitors include apixaban, edoxaban, otamixaban, rivaroxaban, and direct thrombin inhibitors include the bivalent hirudins (bivalirudin, lepirudin, desirudin), and the monovalent compounds argatroban and dabigatran.

As blood clotting is a biological mechanism to stop bleeding, a side effect of anticoagulant therapy may be unwanted bleeding events. It is therefore desirable to provide an antidote to be able to stop such anticoagulant-related bleeding events when they occur (Zikria and Ansell, Current Opinion in Hematology 2009, 16(5): 347-356). One way to achieve this is by neutralizing the activity of the anticoagulant compound present in the patient after administration.

Dabigatran etexilate mesylate, is a potent thrombin inhibitor which can be used for example for the post-operative prevention of deep vein thromboses and in stroke prevention, particularly for preventing strokes in patients with atrial fibrillation. Dabigatran Etexilate, which is also referred to as “BIBR 1048” is a synthetic, non-peptide competitive, rapidly acting oral direct thrombin inhibitor. The IUPAC name of dabigatran etexilate is 3-[(2-{[4-(hexyloxycarbonylamino-imino-methyl)-phenylamino]-methyl}-1-methyl-1H-benzimidazole -5-carbonyl)-pyridine-2-yl-amino]-propionic acid ethyl ester and its chemical structure is shown below as Formula-I:

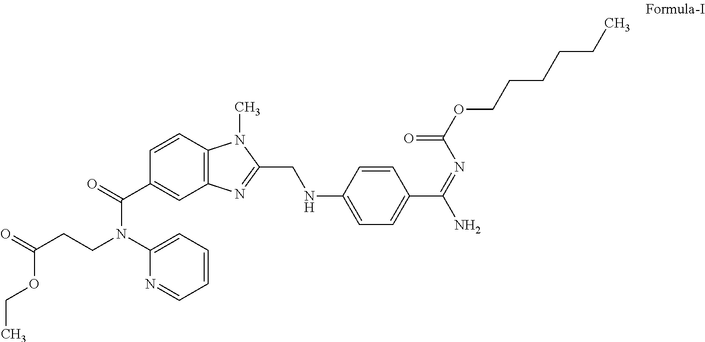 [SMILES: CCCCCCOC(=O)/N=C(/N)c1ccc(NCc2nc3cc(C(=O)N(CCC(=O)OCC)c4ccccn4)ccc3n2C)cc1]

Dabigatran etexilate is currently marketed as Pradaxa® immediate release capsule to reduce the risk of stroke and systemic embolism in patients with non-valvular atrial fibrillation. This formulation contains dabigatran etexilate in the form of the mesylate salt. Each PRADAXA® capsule contains the following inactive ingredients: acacia, dimethicone, hypromellose, hydroxypropylcellulose, tartaric acid, carragenan, potassium chloride, talc, titanium dioxide, and gelatin.

The solubility of the active ingredient in water is only 1.8 mg/ml. Moreover, the active ingredient has a strong pH-dependent solubility that is greatly increased in the acidic environment. This leads to the problem that conventional oral pharmaceutical compositions have large variations in the bioavailability since the solubility of the active ingredient depends on the pH value in the patient's stomach. This is particularly problematic with patients in whom the stomach pH value is changed by physiological variability, illness, or pre medications (for example, PP inhibitors). There is therefore a need for oral pharmaceutical compositions of the active ingredient dabigatran etexilate that provide a release that is independent from the pH value of the stomach and thus, provide bioavailability of the active ingredient.

Dabigatran etexilate is reported in the U.S. Pat. No. 6,087,380 in which the process for the preparation of dabigatran etexilate is disclosed in the example 49, 58a, and example 59. US2006/0183779 patent publication discloses a pharmaceutical composition for oral application that comprises in addition to the active ingredient one or more pharmaceutically acceptable organic acids having as water solubility of >1 g/250 ml at 20.degree. C. However, the corresponding pharmaceutical compositions may cause incompatibilities in the patient. Moreover, the addition of the organic acid restricts the possible amount of active ingredient in an appropriate tablet or capsule. This problem is further exacerbated by the fact that, as a rule, organic acids have only a low buffer capacity so that relatively large amounts of acid have to be added to cause a possible effect on the pH value of the ambience in dissolution of an appropriate tablet.

US 2011/0129538 patent publication discloses a process characterized by a series of partial steps. First, the core is produced from a pharmaceutically acceptable organic acid, preferably tartaric acid by powder layering. The cores are then converted into so-called isolated tartaric acid cores by spraying on an isolating suspension. A dabigatran etexilate suspension prepared subsequently is sprayed onto these coated cores in one or more process steps by means of a coating process. Dabigatran etexilate methanesulfonate, as polymorph I, is suspended together with talc and hydroxypropylcellulose in isopropanol (isopropyl alcohol, 2-propanol, 2-PrOH); the preparation of the suspension being carried out at a temperature not exceeding 30° C. Finally, the active substance pellets thus obtained are packed into suitable capsules.

US 2012/0276206 patent publication discloses a composition of dabigatran etexilate mesylate and use of insulated tartaric acid pellets wherein dabigatran etexilate is layered on the insulated tartaric acid pellets and wherein the insulating layer comprises hydroxypropyl methylcellulose and dimethyl polysiloxane that is added and dissolved therein with stirring, and then talc is added and suspended. This insulating layer prevents the interaction of tartaric acid and dabigatran etexilate mesylate. US 2012/0301541 patent publication discloses the use of compressed cores for pharmaceutical compositions and dosage forms. The compressed cores contain an organic acid, and dabigatran etexilate mesylate salt, and are particularly useful for the preparation of pharmaceutical compositions containing a drug in which dissolution of the drug is favored in acidic environments.

US 2013/0052262 patent publication discloses an oral pharmaceutical composition comprising dabigatran etexilate mesylate salt and inorganic acid excipients such as hydrochloric acid, sulfuric acid, and phosphoric acid. US 2013/0177652 patent publication discloses process for the preparation of a solid oral dosage form comprising dabigatran etexilate or a salt thereof as active substance and comprising a spherical core, wherein (a) the spherical core is coated with a solution of tartaric acid and optionally a binder and/or further inert pharmaceutical excipients without powder layering of tartaric acid, and (b) the coated core of step (a) is coated with further layers wherein at least one of the further layers is a layer comprising the active substance.

PCT publication WO 2012/001156 discloses a solid oral dosage form of dabigatran etexilate, in particular dabigatran etexilate methanesulfonate, is produced by suspension/solution layering of tartaric acid onto spherical cores, such as neutral cores comprised of sucrose, microcrystalline cellulose and starch, or tartaric acid pellets, followed by an isolating layer and the layer comprising the active pharmaceutical ingredient. Optionally, an overcoat can be applied. PCT publication WO 2003/74056 discloses in particular example 3 the preparation of dabigatran etexilate mesylate. The resulting product is obtained in a crystalline form characterized by a melting point of 178-179° C. PCT publication WO 2005/028468 discloses two different anhydrous crystalline forms and a hydrated form of dabigatran etexilate mesylate. The crystalline anhydrous forms include Form I, having a melting point of 180±3° C.; and Form II, having a melting point of 190±3° C. The hydrated form has a melting point of 120±5° C. PCT publication WO 2011/110876 describes in example 15 the preparation of dabigatran etexilate mesylate Form IV characterized by a specific powder X-ray diffraction pattern and by having a melting point of 167-169° C. PCT publication WO 2011/110478 discloses two different non-crystalline forms of dabigatran etexilate mesylate: a solid solution comprising a solid solvent and dabigatran etexilate mesylate dissolved therein; and an amorphous form of dabigatran etexilate mesylate as a composition with one or more hydrophilic polymers, wherein the hydrophilic polymer stabilizes the amorphous state of the active ingredient particles. PCT publication WO 2013/124340 discloses compositions comprising a mixture of at least two types of particles wherein a) the first type of particles comprise dabigatran etexilate in the form of the free base or in the form of pharmaceutically acceptable salts, polymorphs, solvates or hydrates thereof; and b) the second type of particles comprise at least one pharmaceutically acceptable organic acid.

It is known from the prior art, that in particular weakly basic drugs and their salts, demonstrate solubilities that are pH-dependent. In standard matrix formulations, such drugs show a decreased release from the matrix once the formulation enters the higher pH environment of the gastrointestinal tract. The result of this is an unacceptably low, and potentially incomplete, release of the drug from the formulation. Dabigatran has a pH-dependent release profile. The prior art teachings had tried to solve the problem. However, still there is a continuing need to provide stable, new and improved dosage forms of drugs that have pH dependent solubilities, such as weakly basic drugs and their salts, including dabigatran. There is further a need to provide simplified and more cost effective processes for the preparation of the dosages forms of such drugs which have desired chemical and polymorphic stability. The present invention addresses this need.

The inventors of the instant invention with expense of intellectual effort and careful experimentation have developed compositions comprising dabigatran which guarantees sufficient bioavailability of the active substance which is better than that obtained with a conventional pharmaceutical preparation and is largely independent of the pH of the stomach, reduces fluctuations in the bioavailability of the active substance and prevents mal-absorption. Further, the compositions of the present invention have comparable dissolution and bioavailability with respect to the marketed product Pradaxa® (Dabigatran etexilate mesylate capsules) Another advantageous property of the pharmaceutical composition according to the invention is its suitability for all patients, i.e., including those whose gastric pH is raised as a result of normal physiological variability, illness, or co-medication with drugs which increase the gastric pH (e.g., pantoprazole).

### SUMMARY OF THE INVENTION

An aspect of the present invention provides stable oral pharmaceutical compositions comprising direct thrombin inhibitors or pharmaceutically acceptable salts, esters, hydrates and solvates thereof as an active agent, and at least one acidic agent(s), optionally with one or more other pharmaceutically acceptable excipient(s).

An aspect of the present invention provides stable oral pharmaceutical compositions comprising dabigatran etexilate or pharmaceutically acceptable salts, esters, hydrates and solvates thereof, as an active agent and at least one acidic agent(s), optionally with one or more other pharmaceutically acceptable excipient(s).

An aspect of the present invention provides stable oral pharmaceutical compositions comprising dabigatran etexilate or pharmaceutically acceptable salts, esters, hydrates and solvates thereof as an active agent, at least one acidic agent(s) selected from group comprising inorganic acid and organic acid or its pharmaceutically acceptable salt(s), optionally with one or more pharmaceutically acceptable excipient(s).

An aspect of the present invention provides stable oral pharmaceutical compositions comprising dabigatran etexilate or pharmaceutically acceptable salts, esters, hydrates and solvates thereof, as an active agent in first part and at least one acidic agent(s) or its pharmaceutically acceptable salt thereof in second part.

In an aspect, the present invention provides process for the preparation of stable oral pharmaceutical compositions, wherein the process comprises of the following steps:
(i) treating dabigatran etexilate or pharmaceutically acceptable salts, esters, hydrates and solvates thereof with at least one acidic agent(s) or its pharmaceutically acceptable salt(s) thereof,
(ii) optionally adding one or more other pharmaceutically acceptable excipient(s), and
(iii) formulating the material of step (i) and (ii) into a suitable dosage form.

An aspect of the present invention relates to method of using such compositions for reducing the risk of stroke and systemic embolism in patients with non-valvular atrial fibrillation and also for the treatment and reducing the risk of deep venous thrombosis (DVT) and pulmonary embolism (PE). An aspect of the present invention relates to method of treating a patient for the reduction of risk of stroke and systemic embolism in patients with non-valvular atrial fibrillation and reduction of deep venous thrombosis (DVT) and pulmonary embolism (PE).

### DETAILED DESCRIPTION OF THE INVENTION

The term “composition” or “pharmaceutical composition” or “dosage form” as used herein synonymously include solid dosage forms such as granules, multiunit particulate systems (MUPS), pellets, spheres, tablets, capsules, mini-tablets, beads, particles and the like; and liquid dosage forms such as solutions, suspensions, emulsions, colloids and the like, meant for oral administration.

The term ‘stable’ refers to formulations that substantially retain the label amount of the therapeutically active ingredient during storage for commercially relevant times, and the drug-related impurity contents in the formulations remain within acceptable limits. Further, the term ‘stable’ also optionally refers to formulations that contain polymorphically stable active ingredient. The phrase “substantially pure polymorphic form of dabigatran etexilate or its salt thereof”, unless otherwise specified is to be understood as a substance free of other polymorphic and/or pseudopolymorphic forms at amounts detectable with typical analytical methods such as X-ray powder diffraction and/or solid state infrared absorption, i.e. containing less than 10% of other polymorphic and/or pseudopolymorphic forms.

The term ‘pharmaceutically acceptable’ as used herein, refers to materials that are suitable for use in contact with the tissues of humans and lower animals without undue toxicity, irritation, allergic response, and the like, in keeping with a reasonable benefit-risk ratio, and effective for their intended use.

The term “dabigatran” unless indicated otherwise, refers to dabigatran in its free base form, or as a prodrug dabigatran etexilate, or as a pharmaceutically acceptable salt, or esters, or hydrates or solvates thereof. Preferably dabigatran is in the form of a prodrug dabigatran etexilate or pharmaceutically acceptable acid addition salt, more preferably, in the form of dabigatran etexilate methanesulfonate (mesylate) salt. According to the present invention, 90% of particles with particle size less than about 100 μm, and/or surface area less that about 5 m²/gm are useful. Particularly according to the present invention, 90% of particles with particle size less than about 80 μm and/or surface area less that about 5 m²/gm are useful.

As used in this specification, the singular forms “a”, “an”, and “the” include plural references unless the context clearly dictates otherwise. Thus for example, a reference to “a process” includes one or more process, and/or steps of the type described herein and/or which will become apparent to those persons skilled in the art upon reading this disclosure and so forth.

In an embodiment, the present invention provides stable oral pharmaceutical compositions comprising direct thrombin inhibitors or pharmaceutically acceptable salts, esters, hydrates and solvates thereof as an active agent, at least one acidic agent(s) or its pharmaceutically acceptable salt(s), optionally with one or more other pharmaceutically acceptable excipient(s).

In an embodiment, the present invention provides stable oral pharmaceutical compositions comprising dabigatran etexilate or pharmaceutically acceptable salts, esters, hydrates and solvates thereof as an active agent, and at least one acidic agent(s), optionally with one or more other pharmaceutically acceptable excipient(s).

In another embodiment, the present invention provides stable oral pharmaceutical compositions comprising dabigatran etexilate or pharmaceutically acceptable salts, esters, hydrates and solvates thereof as an active agent, at least one acidic agent(s) selected from group comprising inorganic acid and organic acid or its pharmaceutically acceptable salt(s), optionally with one or more pharmaceutically acceptable excipient(s).

In one of the embodiments, the present invention provides stable oral pharmaceutical compositions comprising dabigatran etexilate or pharmaceutically acceptable salts, esters, hydrates and solvates thereof as an active agent from about 0.1% w/w to about 99% w/w of the composition, acidic agent(s) or its pharmaceutically acceptable salt(s) from about 0.1% to about 99% w/w of the composition, optionally with one or more pharmaceutically acceptable excipient(s), from about 0.9% to about 97% w/w of the composition.

In an embodiment, the present invention provides stable oral pharmaceutical compositions comprising dabigatran etexilate or pharmaceutically acceptable salts, esters, hydrates and solvates thereof as an active agent from about 0.1% w/w to about 99% w/w of the composition, acidic agent(s) selected from group comprising inorganic acid and organic acid or its pharmaceutically acceptable salt(s) from about 0.1% to about 99% w/w of the composition, optionally with one or more pharmaceutically acceptable excipient(s), from about 0.9% to about 97% w/w of the composition.

In an embodiment the present invention provides stable oral pharmaceutical compositions, wherein the ratio of active agent(s) to acidic agent(s) is from about 0.1:100 to about 100:0.1. Preferably, ratio of active agent(s) to acidic agent(s) is from about 0.1:50 to about 50:0.1, more preferably ratio of active agent(s) to acidic agent(s) is from about 0.1:20 to about 20:0.1,

In an embodiment the present invention provides stable oral pharmaceutical compositions, comprising dabigatran etexilate as an active agent(s) or pharmaceutically acceptable salts, esters, hydrates and solvates thereof as an active agent, additionally along with at least one another active agent. In an embodiment, the said at least one another active agent complements the pharmacological use of dabigatran or is useful in treating any associated disease conditions.

In an embodiment the present invention provides stable oral pharmaceutical compositions comprising comprising dabigatran etexilate or pharmaceutically acceptable salts, esters, hydrates and solvates thereof as an active agent in first part, and at least one acidic agent(s) selected from group comprising inorganic acid and organic acid or its pharmaceutically acceptable salt thereof in second part.

In another embodiment of the present invention, the first part comprising dabigatran etexilate or pharmaceutically acceptable salts, esters, hydrates and solvates thereof is in the form of mini-tablets having a diameter more than about 1 mm., preferably between about 1 mm. to about 6 mm.

In an embodiment, the present invention provides process for the preparation of stable oral pharmaceutical compositions, wherein the process comprises of the following steps:
(i) treating dabigatran etexilate or pharmaceutically acceptable salts, esters, hydrates and solvates thereof with acidic agent(s) or its pharmaceutically acceptable salt(s) thereof,
(ii) optionally adding one or more other pharmaceutically acceptable excipient(s), and
(iii) formulating the material of step (i) and (ii) into a suitable dosage form

In an embodiment, the present invention provides process for the preparation of stable oral pharmaceutical compositions, wherein the process comprises of the following steps:
(i) treating dabigatran etexilate or pharmaceutically acceptable salts, esters, hydrates and solvates thereof with acidic agent(s) selected from group comprising inorganic acid and organic acid or its pharmaceutically acceptable salt(s) thereof,
(ii) optionally adding one or more other pharmaceutically acceptable excipient(s), and
(iii) formulating the material of step (i) and (ii) into a suitable dosage form

In one of the embodiments is provided process for the preparation of stable oral pharmaceutical compositions, wherein the process comprises of the following steps:
(i) treating dabigatran etexilate with tartaric acid or its pharmaceutically acceptable salt(s),
(ii) optionally adding one or more pharmaceutically acceptable excipient(s), and
(iii) compressing the granules to form a tablet or filling into capsules.

In another embodiment is provided process for the preparation of stable oral pharmaceutical compositions, wherein the process comprises of the following steps:
i) mixing dabigatran etexilate or pharmaceutically acceptable salts, esters, hydrates and solvates thereof one or more other pharmaceutically acceptable excipient(s),
ii) compressing the blend of step (i) to form tablets,
iii) optionally coating the tablets of step (ii) with suitable coating agent(s), and
iv) filling tablets of step (ii) or step (iii) along with tartaric acid or its pharmaceutically acceptable salt(s) thereof into a capsule.

In one of the embodiments is provided process for the preparation of stable oral pharmaceutical compositions, wherein the process comprises of the following steps:
i) preparing dispersion of dabigatran etexilate or pharmaceutically acceptable salts, esters, hydrates and solvates thereof in one or more solvent(s),
ii) coating pellets comprising one or more other pharmaceutically acceptable excipient(s) with dispersion of step (i),
iii) optionally further coating pellets of step (ii) with suitable coating agent(s), and
iv) filling pellets of step (ii) or step (iii) along with tartaric acid or its pharmaceutically acceptable salt(s) thereof into a capsule.

As used herein, unless indicated otherwise, references to total weight of the pharmaceutical composition refers to the total weight of the active agent(s) and pharmaceutically acceptable excipient(s).

In an embodiment, the inorganic acids are selected from but not limited to group comprising hydrochloric acid, sulfuric acid, and phosphoric acid and the like, or their pharmaceutically acceptable salt(s); and organic acids are selected from but not limited to group comprising lactic acid, citric acid, tartaric acid, malic acid, maleic acid, mandelic acid and the like or their pharmaceutically acceptable salt(s). Preferably, the organic acid is a tartaric acid or its pharmaceutically acceptable salt.

“Pharmaceutically acceptable excipient(s)” are components other than the active ingredient that are added to make a pharmaceutical formulation. Excipients may be added to facilitate manufacture, enhance stability, enhance product characteristics, enhance bioavailability, enhance patient acceptability, etc. Useful pharmaceutical excipients according to the present invention include diluents, binders, disintegrants, surfactants, glidant, lubricants, glidants/antiadherants; chelating agents; vehicles; bulking agents; stabilizers; preservatives and a combination thereof. It will be appreciated by the person skilled in the art that a particular excipient may act as both a binder and filler, or as a binder, filler and a disintegrant, or can have any other uses.

Exemplary “diluents” include, but are not limited to microcrystalline cellulose, lactose, sugar, starches, modified starches, pregelatinized starch, talc, kaolin, sucrose, mannitol, sorbitol, dextrates, dextrin, maltodextrin, dextrose, sorbitol, xylitol, lactitol, calcium carbonate, calcium sulfate, dibasic calcium phosphate, tribasic calcium phosphate, magnesium carbonate, magnesium oxide and the like used either alone or in combinations thereof. Exemplary “binders” include, but are not limited to, hydroxypropyl cellulose, hydroxypropyl methylcellulose, povidone, starches such as corn starch, potato starch, modified starches, sugars, guar gum, pectin, wax binders, methylcellulose, carboxymethylcellulose, hydroxyethyl cellulose, copolyvidone, carboxymethylcellulose sodium, ethyl cellulose, gelatin, liquid glucose, pregelatinized starch, sodium alginate, acacia, alginic acid, tragacanth, and the like, used either alone or in combinations thereof.

Disintegrants according to the present invention are selected from, but not limited to, cellulose and its derivatives including low-substituted hydroxypropyl cellulose; cross-linked polyvinylpyrrolidone; cross-linked sodium carboxymethylcellulose, sodium carboxymethylcellulose, microcrystalline cellulose; sodium starch glycolate; ion-exchange resins; starch and modified starches including pregelatinized starch; formalin-casein and the like used either alone or in combinations thereof.

Exemplary “glidants” include, but are not limited to, colloidal silica, calcium silicate, magnesium silicate, silicon hydrogel, cornstarch, talc, corn starch, DL-leucine and the like used either alone or in combinations thereof. Exemplary “lubricants” include, but are not limited to, magnesium stearate, calcium stearate, sodium stearyl fumarate, zinc stearate, stearic acid, fumaric acid, palmitic acid, talc, carnauba wax, hydrogenated vegetable oils, mineral oil, polyethylene glycols and the like, used either alone or in combinations thereof.

Exemplary “surfactants” include, but are not limited to, sodium lauryl sulfate, polyethylene glycols, polyethylene glycol fatty acid esters such as PEG monolaurate, PEG dilaurate, PEG distearate, PEG dioleate; polyoxyethylene alkylaryl ethers such as polyoxyethylene lauryl ether, polyoxyethylene acetyl ether, polyoxyethylene stearyl ether; polyoxyethylenesorbitan fatty acid ester such as polysorbate 40, polysorbate 60, polysorbate 80; sorbitan fatty acid mono esters such as sorbitan monolaurate, sorbitan monooleate, sorbitan sesquioleate, sorbitan trioleate, poloxamers, polyoxyethylene castor oil derivates such as polyoxyl castor oil, polyoxyl hydrogenated castor oil and the like used either alone or in combinations thereof. Exemplary “granulating solvents” but are not limited to, purified water, isopropyl alcohol, dichloromethane, ethanol, acetone, methylene chloride and the like, used either alone or in combinations thereof.

In an embodiment of the present invention, the composition may additionally comprise a conventionally known antioxidant such as ascorbyl palmitate, butyl hydroxy anisole, butyl hydroxy toluene, propyl gallate and alpha-tocopherol.

It must be appreciated that the pharmaceutical compositions of the present invention can include all the dosage forms known to a person skilled in art, viz. formulations such as single unit dosage forms in the form of tablets, bilayer tablets, inlaid tablets, tablet in tablet, multilayered tablets, minitablets filled in capsules and the like; beads, pellets presented in a sachet, capsule or tablet capsules such as soft and hard gelatin; lozenges or sachets; granulates, microparticles, multiparticulates, powder and the like. In an embodiment, the pharmaceutical composition of the present invention can be prepared by either direct compression, dry compression (slugging), or by granulation, preferably by granulation. The granulation technique is either aqueous or non-aqueous. In an embodiment, the equipment used for the granulation are selected from but not limited to rapid mixer granulators, fluidized bed granulators or the like. It must be appreciated that person skilled in the art would know the possible variations and modifications in the type of equipment used in the manufacturing process and are within the scope of the instant invention.

In an embodiment, the tablet compositions of the present invention may be film coated. A film forming agent may provide smooth film-forming coating suspensions and enhance the rheological mechanical strength properties of film coating gel matrices. Film forming agents include, for example, polyvinyl pyrrolidone, natural gums, starches, poly vinyl acetate based and cellulosic polymers. A cellulosic polymer may include a molecule comprising at least one cellulose polymer or derivative modified with small amounts of propylene glycol ether groups attached to the cellulose anhydroglucose chain affording binding properties that enhance the reinforcing film properties of film applications. Examples of cellulosic polymers include, but are not limited to, hydroxypropyl methyl cellulose (“HPMC”), carboxymethyl cellulose (“CMC”) or salts thereof, hydroxypropyl cellulose (“HPC”), methylcellulose (“MC”), hydroxyethyl cellulose (“HEC”), and the like. In addition, cellulosic polymers may be characterized as ionic or non-ionic. Ionic cellulosic polymers include, for example, sodium CMC. Non-ionic cellulosic polymers include, for example, HPMC, HPC, HEC, and MC. A variety of commercially available cellulosic polymers exists and may include, for example, Spectracel® HPMC compositions (available from Sensient Technologies).

In an embodiment, the compositions of the present invention may additionally comprise of a colorant in order to produce a desirable color. Colors known to be ‘FD&C’ certified may be used to provide coloring to the product and are within the purview of the present invention. Suitable colorants include natural colorants i.e., pigments and dyes obtained from mineral, plant, and animal sources. Examples of natural colorants include red ferric oxide, yellow ferric oxide, annattenes, alizarin, indigo, rutin, quercetin, and the like. Synthetic colorants may also be used, which is typically an FD&C or D&C dye, e.g., an approved dye selected from the so-called ‘coal-tar’ dyes, such as a nitroso dye, a nitro dye, an azo dye, an oxazine, a thiazine, a pyrazolone, a xanthene, an indigoid, an anthraquinone, an acridine, a rosaniline, a phthalein, a quinoline, or a ‘lake’ thereof, i.e. an aluminum or calcium salt thereof. Particularly preferred colorants are food colorants in the ‘GRAS’ (Generally Regarded as Safe) category.

In another embodiment, the present invention provides method of using such compositions for reducing the risk of stroke and systemic embolism in patients with non-valvular atrial fibrillation.

The compositions of the present invention can be packed into suitable containers such as bottles, blisters or pouch. Further, the packages may optionally contain a dessicant or an antioxidant or oxygen absorbant or combinations thereof.

The following examples serve to illustrate the embodiments of the present invention. However, they do not intend to limit the scope of the invention. It is obvious to those skilled in the art to find out the composition for other dosage forms and substitute the equivalent excipients as described in this specification or with the one known to the industry.

### EXAMPLE 1

| S. No. | Ingredients | % w/w |
|---|---|---|
| | Intragranular | |
| 1 | Dabigatran etexilate mesylate | 40.22 |
| 2 | Microcrystalline cellulose | 12.00 |
| 3 | Croscarmellose sodium | 4.50 |
| 4 | Colloidal silicon dioxide | 1.00 |
| 5 | Purified water* | q.s. |
| | Extragranular | |
| 6 | Citric acid | 38.00 |
| 7 | Hydroxypropyl methylcellulose | 3.28 |
| 8 | Magnesium stearate | 1.00 |

*Lost in processing

### Manufacturing Process

i) Dabigatran etexilate mesylate, croscarmellose sodium, microcrystalline cellulose and colloidal silicon dioxide were sifted together.
ii) The blend of step (i) was granulated with water as and was dried.
iii) The granules obtained in step (ii) were blended with citric acid and hydroxypropyl methylcellulose.
iv) The granules of step (iii) were lubricated with magnesium stearate.
v) The lubricated granules of step (iv) were filled into capsules.

### EXAMPLE 2

| S. No. | Ingredients | % w/w |
|---|---|---|
| | Dabigatran granules | |
| 1 | Dabigatran etexilate mesylate | 40.22 |
| 2 | Lactose monohydrate | 15.00 |
| 3 | Pregelatinized starch | 3.50 |
| 4 | Silicon dioxide | 1.00 |
| 5 | Purified water* | q.s. |
| | Tartaric acid granules | |
| 6 | Tartaric acid | 38.00 |
| 7 | Dibasic calcium phosphate | 1.28 |
| 8 | Purified water * | q.s. |
| 9 | Zinc stearate | 1.00 |
| | Film coating | |
| 10 | Opadry ® white | 3.00 |

*Lost in processing

### Manufacturing Process

i) Dabigatran etexilate mesylate, lactose monohydrate, pregelatinized starch and silicon dioxide were sifted together.
ii) The blend of step (i) was granulated with purified water and dried.
iii) Tartaric acid and dibasic calcium phosphate were sifted and granulated with purified water.
iv) The granules of step (ii) and step (iii) were blended and lubricated with zinc stearate.
v) The lubricated granules of step (iv) were compressed into tablets by using a suitable compression machine.
vi) The tablets of step (v) were coated with Opadry coating solution to obtain the desired weight gain.

### EXAMPLES 3A, 3B & 3C

| | | Example | Example | Example |
|---|---|---|---|---|
| | | 3A | 3B | 3C |
| S. No. | Ingredients | (% w/w) | (% w/w) | (% w/w) |
| | Tablet portion | | | |
| 1 | Dabigatran etexilate mesylate | 40.22 | 40.22 | 40.22 |
| 2 | Microcrystalline cellulose | 11.95 | 11.96 | 10.34 |
| 3 | Hydroxypropyl cellulose | 4.65 | 4.65 | 4.65 |
| 4 | Croscarmellose sodium | 3.05 | 3.02 | 4.65 |
| 5 | Magnesium stearate | 0.60 | 0.60 | 0.60 |
| 6 | Isopropyl Alcohol | — | — | q.s |
| | Film coating | | | |
| 7 | Opadry Coating | — | 3.00 | 1.82 |
| 8 | Purified water* | — | q.s. | q.s |
| | Pellet Portion | | | |
| 9 | Tartaric acid pellets | 39.53 | 36.37 | 37.72 |
| 10 | Colloidal silicon dioxide | — | 0.18 | — |

*Lost in processing

### Manufacturing Process

### Example-3A

i) Dabigatran etexilate mesylate, microcrystalline cellulose, hydroxypropyl cellulose and croscarmellose sodium were sifted together.
ii) The blend of step (i) was lubricated with magnesium stearate.
iii) The lubricated blend of step (ii) was compressed into mini tablets.
iv) 4 mini tablets of step (iii) and tartaric acid pellets were filled into capsules.

### Example 3B

i) Dabigatran etexilate mesylate, microcrystalline cellulose, hydroxypropyl cellulose and croscarmellose sodium were sifted together.
ii) The blend of step (i) was lubricated with magnesium stearate.
iii) The lubricated blend of step (ii) was compressed into mini tablets.
iv) The compressed mini tablets of step (iii) were film coated with Opadry coating dispersion.
v) Tartaric acid pellets were blended with colloidal silicon dioxide
vi) 4 film coated mini tablets of step (iv) along with blend of step (v) were filled into capsules.

### Example 3C

i) Dabigatran etexilate mesylate, microcrystalline cellulose, hydroxypropyl cellulose and croscarmellose sodium were sifted together.
ii) The blend of step (i) was granulated with isopropyl alcohol to form granules.
iii) The granules of step (ii) were lubricated with magnesium stearate.
iv) The lubricated blend of step (iii) was compressed into mini tablets.
v) The compressed mini tablets of step (iv) were film coated with Opadry coating dispersion.
vi) 4 mini tablets of step (v) and tartaric acid pellets were filled into capsules.

### EXAMPLES 4A, 4B & 4C

| | | Example | Example | Example |
|---|---|---|---|---|
| | | 4A | 4B | 4C |
| S. No. | Ingredients | (% w/w) | (% w/w) | (% w/w) |
| | Pellets coated with drug | | | |
| 1 | Microcrystalline cellulose pellets | 20.83 | 20.83 | 20.83 |
| | Drug Suspension | | | |
| 2 | Dabigatran etexilate mesylate | 40.22 | 40.22 | 40.00 |
| 3 | Hydroxypropyl cellulose | 5.79 | — | 8.11 |
| 4 | Povidone | — | 6.00 | — |
| 5 | Talc | 4.62 | 4.62 | — |
| 6 | Isopropyl alcohol* | — | q.s. | q.s. |
| 7 | Acetone * | q.s | — | — |
| 8 | Opadry ® | — | — | 3.48 |
| 9 | Purified water* | q.s. | q.s. | q.s. |
| | Acid Pellets | | | |
| 10 | Tartaric acid pellets | 25.00 | 25.00 | 27.00 |
| 11 | Colloidal silicon dioxide | 0.13 | 0.13 | 0.46 |

*Lost in processing

### Manufacturing Process

### Drug Pellets

i) Hydroxypropylcellulose/povidone was dispersed in acetone/isopropyl alcohol.
ii) Dabigatran etexilate mesylate and talc were added to the dispersion of step (i) to form a uniform dispersion.
iii) Microcrystalline cellulose pellets were coated with dispersion of step (ii).
iv) The drug loaded pellets of step (iii) were optionally film coated with Opadry® AMB dispersion.
v) Tartaric acid pellets were blended with colloidal silicon dioxide.
vi) The drug loaded pellets of step (iv) and tartaric acid pellets of step (v) were filled into capsule or compressed into tablets using a suitable compression machine.

### Dissolution Data:

Dissolution studies of dabigatran etexilate mesylate capsules of example 4A, 4B, 4C and Reference product (Pradaxa®) were carried with USP-1 (Basket with modified diameter of 24.5 mm) at 100 rpm using 900 ml 0.01 N HCl.

| | Example 4A | Example 4B | Example 4C | Pradaxa ® |
|---|---|---|---|---|
| Time | Percent drug | Percent drug | Percent drug | Percent drug |
| (min) | release | release | release | release |
| 10 | 58 | 72 | 56 | 34 |
| 15 | 85 | 91 | 87 | 86 |
| 20 | 96 | 96 | 98 | 92 |
| 30 | 98 | 99 | 100 | 99 |
| 45 | 102 | 99 | 100 | 99 |

### Stability Data:

The dabigatran etexilate mesylate capsules of example 4C were packed in HDPE bottles and subjected to stability testing at 40° C.±2° C./75% RH±5% RH for 1 month and 3 months.

| | Example 4C | | |
|---|---|---|---|
| Condition | Initial | 40° C./75% RH-1M | 40° C./75% RH-3M |
| Assay | 101.7 | 100.5 | 100.1 |
| Dabigatran Acid | 0.00 | 0.00 | 0.00 |
| Dabigatran Ethyl Ester | 0.01 | 0.02 | 0.04 |
| Dabigatran Hexilate | 0.11 | 0.10 | 0.12 |
| Dabigatran Etexilate | 0.11 | 0.14 | 0.15 |
| amide | | | |
| Any Unknown impurity | 0.09 | 0.08 | 0.06 |
| Total Impurity | 0.49 | 0.50 | 0.55 |

### EXAMPLE 5A, 5B & 5C

| | | Example | Example | Example |
|---|---|---|---|---|
| | | 5A | 5B | 5C |
| S. No. | Ingredients | (% w/w) | (% w/w) | (% w/w) |
| | Tablet | | | |
| 1 | Dabigatran etexilate mesylate | 40.22 | 40.03 | 40.22 |
| 2 | Microcrystalline cellulose | 10.33 | 17.74 | 16.00 |
| 3 | Hydroxypropyl cellulose low | 4.65 | 1.80 | — |
| | substituted | | | |
| 4 | Hydroxypropyl methylcellulose | — | 6.01 | — |
| 5 | Povidone K-30 | — | — | 1.16 |
| 6 | Povidone K-90 | — | — | 2.32 |
| | Binder solution | | | |
| 7 | Isopropyl alcohol* | q.s. | — | — |
| | Extragranular | | | |
| 8 | Croscarmellose sodium | 4.65 | — | — |
| 9 | Magnesium sterate | 0.60 | 0.60 | 0.60 |
| 10 | Opadry ® AMB | 1.81 | — | — |
| 11 | Purified water* | q.s. | — | — |
| | Acid Pellets | | | |
| 12 | Tartaric acid pellets | 37.72 | 33.80 | 39.53 |

*Lost in processing

### Manufacturing Process

i) Dabigatran etexilate mesylate, microcrystalline cellulose, hydroxypropyl cellulose low substituted/hydroxypropyl methylcellulose and povidone K30/povidone K90 were sifted together.
ii) The blend of step (i) was granulated with isopropyl alcohol/slugged/compacted and milled.
iii) The granules obtained in step (ii) were blended with croscarmellose sodium.
iv) The blend of step (iii) was lubricated with magnesium stearate.
v) The lubricated granules of step (iv) was compressed into tablets.
vi) The compressed tablets were film coated with Opadry AMB dispersion.
vii) The film coated tablets of step (vi)/tablets of step (v) and tartaric acid pellets were filled into capsules.

We claim:

1. A stable oral pharmaceutical composition comprising dabigatran etexilate or pharmaceutically acceptable salts, esters, hydrates and solvates thereof as an active agent, at least one acidic agent and optionally at least one pharmaceutically acceptable excipient.

2. The composition according to claim 1, wherein acidic agent is selected from group consisting of inorganic acid, organic acid and pharmaceutically acceptable salt thereof.

3. The composition according to claim 1, wherein the said active agent is from about 0.1% w/w to about 99% w/w of the composition, the said acidic is from about 0.1% to about 99% w/w of the composition, and the said pharmaceutically acceptable excipient is from about 0.9% to about 97% w/w of the composition.

4. The composition according to claim 2, wherein inorganic acid is selected from group consisting of hydrochloric acid, sulfuric acid, phosphoric acid and pharmaceutically acceptable salt thereof.

5. The composition according to claims 2, wherein organic acid is selected from group consisting of lactic acid, citric acid, tartaric acid, malic acid, maleic acid, mandelic acid and pharmaceutically acceptable salt thereof.

6. The composition according to claim 1, wherein the pharmaceutically acceptable excipient is selected from a group consisting of diluents, binders, disintegrants, surfactants, glidants, lubricants, glidants, antiadherants, chelating agents, vehicles, bulking agents, stabilizers, preservatives and combinations thereof.

7. The compositions according to claim 1, wherein the ratio of said active agent to said acidic agent is from about 0.1:100 to about 100:0.1.

8. The composition according to claim 1, comprising dabigatran etexilate or pharmaceutically acceptable salts, esters, hydrates and solvates thereof as an active agent in first part, and at least one acidic agent(s) selected from group comprising inorganic acid and organic acid or its pharmaceutically acceptable salt thereof in second part.

9. The composition according to claim 8, wherein first part is in the form of mini-tablets having a diameter more than about 1 mm.

10. A process for the preparation of stable oral pharmaceutical composition according to claim 1, wherein the process comprises of the following steps:
i) treating dabigatran etexilate or pharmaceutically acceptable salts, esters, hydrates and solvates thereof with at least one acidic agent or its pharmaceutically acceptable salt(s) thereof,
ii) optionally adding one or more other pharmaceutically acceptable excipient(s), and
iii) formulating the material obtained from step (i) or (ii) into a suitable dosage form.

11. A process according to claim 10, wherein the said acidic agent is either an inorganic acid or an organic acid.

12. A process for the preparation of stable oral pharmaceutical composition according to claim 1, wherein the process comprises of the following steps:
i) blending dabigatran etexilate or pharmaceutically acceptable salts, esters, hydrates and solvates thereof with at least one pharmaceutically acceptable excipient,
ii) compressing the blend of step (i) to form tablets,
iii) optionally coating the tablets of step (ii) with suitable coating agent, and
iv) filling tablets from step (ii) or step (iii) along with tartaric acid or its pharmaceutically acceptable salt(s) thereof into a capsule.

13. A process for the preparation of stable oral pharmaceutical composition according to claim 1, wherein the process comprises of the following steps:
i) Preparing a dispersion of dabigatran etexilate or pharmaceutically acceptable salts, esters, hydrates and solvates thereof in at least one solvent,
ii) coating pellets with a composition comprising the dispersion of step (i) and at least one pharmaceutically acceptable excipient,
iii) optionally further coating pellets of step (ii) with suitable coating agent, and
iv) filling pellets of step (ii) or step (iii) along with at least one acidic agent or pharmaceutically acceptable salt thereof into a capsule.

14. A treatment method for reducing the risk of stroke and systemic embolism in patients with non-valvular atrial fibrillation or for reduction of deep venous thrombosis(DVT) and pulmonary embolism (PE) comprising the administration of an effective amount of the composition of claim 1.
